(19) United States
(12) Patent Application Publication
(10) Pub. No.: US 2019/0343997 A1
(43) Pub. Date: Nov. 14, 2019

(54) BLOOD PUMP

(71) Applicant: Berlin Heart GmbH, Berlin (DE)

(72) Inventors: Marcus Granegger, Berlin (DE); Kim Peter Winterwerber, Berlin (DE)

(73) Assignee: Berlin Heart GmbH, Berlin (DE)

(21) Appl. No.: 16/336,513

(22) PCT Filed: Sep. 29, 2017
(86) PCT No.: PCT/EP2017/074796

§ 371 Date: Mar. 26, 2019

(30) Foreign Application Priority Data
Sep. 29, 2016 (EP) 16191613.5

(51) Int. Cl. A61M 1/10; A61M 1/12

(52) U.S. Cl. CPC A61M 1/1015; A61M 1/122; F04D 29/0467; A61M 1/1031; A61M 1/1017

(57) ABSTRACT

The present application relates to a blood pump having a mechanical bearing assembly. A ball segment is provided which is made of a diamond.

### CROSS-REFERENCE TO RELATED APPLICATIONS

This application is a 371 nationalization of international patent application PCT/EP2017/074796 filed Sep. 29, 2017, which claims priority under 35 USC § 119 to European patent application EP 16191613.5 filed Sep. 29, 2016. The entire contents of each of the above-identified applications are hereby incorporated by reference.

### TECHNICAL FIELD

The present invention relates to a blood pump and to a bearing assembly for a blood pump.

### BRIEF DESCRIPTION OF THE DRAWINGS

FIG. 1 shows a schematic depiction of an axial pump;

FIG. 2 shows a schematic depiction of an axial pump with a tangential outlet;

FIG. 3 shows a schematic depiction of a further blood pump;

FIG. 4 shows a variant of a mechanical bearing;

FIG. 5 shows a further variant of a mechanical bearing;

FIG. 6 shows an illustration of a contact angle;

FIG. 7 shows a further embodiment of a bearing assembly;

FIG. 8 shows a further embodiment of a bearing assembly; and

FIG. 9 shows a further embodiment of a bearing assembly.

### DETAILED DESCRIPTION

Various possibilities for supporting a rotor in blood pumps are known in the prior art. The most conventional are mechanical, magnetic or hydrodynamic bearings, or mixed forms of the aforementioned bearings.

In the case of mechanical bearings, what are known as “ball/cup” bearings are known. For example, U.S. Pat. No. 5,707,218 describes bearings of this kind, in which a ball segment is mounted both axially and radially in a cap segment.

Mechanical bearings of this kind have numerous disadvantages on account of the high contact forces.

The object of the present invention is to provide a bearing assembly for a blood pump which overcomes the present disadvantages.

The blood pump comprises a housing with an upstream inlet, a downstream outlet, and a rotatably mounted rotor with an axis and a blading. A mechanical bearing for supporting the rotor is also provided, wherein the mechanical bearing comprises at least one ball segment and at least one cap segment receiving the ball segment at least in part. A ball segment is understood here to mean a spherical cap which is either a sphere or part of a sphere formed by cutting through the sphere in one plane. The ball segment is pressed onto the cap segment such that a form-locked connection is provided between the two elements.

A cap segment is understood here to mean the surface of the interior of a spherical cap, i.e. a spherical dome, or the interior of a spherical zone. A spherical dome forms the curved part of the surface of a ball segment. The spherical zone forms the curved surface part of a spherical segment.

In the case of the cap segment, it must be ensured that the height of the cap segment along the axis of rotation is not greater than a corresponding radius of the ball. The ball segment and the cap segment are rotationally symmetrical about an axis of rotation, at least in sections, i.e. the surfaces of the ball segment and of the cap segment resting against one another or pressed against one another are rotationally symmetrical. Here, the axis of rotation is substantially coincident with an axis of the rotor.

The ball segment consists of a hard material, preferably produced by a chemical deposition process. Hard materials are understood here to mean, for example, diamonds, metal carbides or certain ceramics. For example, besides diamonds, nitrides may also be included under the term hard materials, such as preferably a crystalline cubic boron nitride, a titanium nitride or a silicon nitride, or carbides, such as preferably a silicon carbide, a boron carbide, tungsten carbide, a vanadium carbide, a titanium carbide, or a tantalum carbide, and oxides, such as preferably an aluminium oxide or a zirconium dioxide. These hard materials have good abrasion properties on account of their hardness. Furthermore, diamonds have a good thermal conductivity, such that the heat created by friction between the ball segment and cap segment can be well taken up by the fluid, for example blood, flowing through the blood pump. The diamonds produced by chemical deposition processes (chemical vapour deposition; CVD), or other of the above-mentioned hard materials, for example silicon carbide or boron nitride, have the advantage that they are much more economical compared to natural diamonds and often have an improved thermal conductivity. Furthermore, by means of modern shaping methods, the ball segment can shaped in accordance with the bearing requirements.

The mechanical bearing preferably takes up the majority of the axial forces acting on the rotor. Some of the radially acting forces are preferably also taken up by the cap segment. The frictional area is defined here by the contact area between the ball segment and the cap segment. Here, however, it should be noted that a region of elastohydrodynamic lubrication or mixed friction, i.e. friction by solids or liquids, exists between the ball segment and the cap segment, with a very thin layer of liquid, for example blood as in the present case, or also a saline solution, being provided in said region. The elastohydrodynamic lubrication differs from a hydrodynamic bearing.

In order to achieve a suitable dimensioning of the frictional areas, it can be provided that the curvature (i.e. the radius) of the ball segment or the cap segment is selected in such a way that the force vector formed from the radially and axially acting force from the ball segments to the cap segment is perpendicular to the frictional area. This may give an indication of the radius of curvature to be selected for the cap or ball segment.

Furthermore, the curvature of the ball segment or of the cap segment can be selected in such a way that the ratio of the size of the axial area of the cap segment (i.e. the area of the projection of the cap segment in a plane perpendicular to the axis of rotation) to the radial area of the cap segment (i.e. the area of the radial projection of the cap segment) is set such that it corresponds to the ratio of the axially effective force to the radially effective force.

The ratio of the operational forces axial/radial (Fa/Fr) preferably defines the angle of a starting tangent αₛₜₐᵣₜ>arctan (Fa/Fr).

The angle of an end tangent is defined preferably by the friction value of the material combination. The following is true: α_{end}>arctan(μ), Self-locking, i.e. the risk of excessive friction, exists below this angle.

The cap is preferably made as flat as possible (i.e. the ratio of radius transverse to the axis of rotation to the height parallel to the axis of rotation is much greater than 1), i.e. the angle of the end tangent is maximised without the ball being able to spring out of the cap when wear occurs.

The ball radius is limited downwardly by the permitted wear and the maximum surface temperature. Since the amount of heat produced is theoretically independent of the ball radius, a small ball will get hotter than a large one.

The forces acting axially and radially on the bearing can be determined for example by flow simulations of a fluid along the rotor, and the curvature or the area of the ball segment and/or of the cap segment can be selected in accordance with the forces determined by the simulation.

In contrast to metals having a diamond coating, such as diamond-like carbon (DLC), a diamond or other hard material produced by means of a chemical deposition process has much better thermal conduction values. Furthermore, a ball segment made of diamond is of such a nature that the entire volume of the ball segment can be used for heat exchange between the friction heat and the fluid. Further embodiments of the pump or the bearing assembly will become clear from the dependent claims.

In a variant of the invention the ball segment is a sphere. In alternative embodiments the ball segment is merely a spherical cap, wherein the spherical cap can have a height between the radius and twice the radius of the sphere, i.e. the ball segment is more than a hemisphere.

The sphere forms the maximum body volume in relation to the body surface area and therefore surface area of the mechanical bearing acting as frictional area. Due to the large volume of the sphere, more heat can be absorbed and frictional heat can be better dissipated to the blood.

In the case of a ball segment, with a height of the ball segment along the axis of rotation with a value between half the radius of the sphere and double the radius of the sphere, costs can be saved compared to the volume of a whole sphere on account of the lower volume of the hard material. Here, however, difficulties are encountered in producing a cut surface that is exactly plane. For example, a hard material produced as a sphere can be divided by an appropriate method into two hemispheres (for example by diamond sawing in the case of a diamond sphere). These hemispheres may then be used as components of a mechanical bearing.

In a further embodiment the cap segment has a radius of curvature between a first radial distance and a second radial distance from the axis of rotation corresponding to a radius of curvature of the ball segment. As a result, the cap segment and the ball segment engage with one another at least in sections in the manner of a ball/cup bearing. For example, the cap segment can be of such a nature that the radius of curvature outside the second distance, i.e. with a radius greater than the second distance, is greater than inside the second distance, for example so as to also enable a flushing, even if only small, of the gap between the cap segment and the ball segment.

In one embodiment the radius of curvature of the ball segment and of the cap segment is selected in such a way that in a longitudinal section of the cap segment considered along the axis of rotation the first distance defines a first point of contact and the second distance defines a second point of contact between the cap segment and ball segment, and a straight line defined by the first and second points of contact has an angle of more than 50°, preferably more than 55°, particularly preferably more than 59°, relative to the axis of rotation. In this exemplary embodiment the radius of curvature between the axis of rotation and the first radial distance for example may be much greater, and therefore a small cavity or recess in the cap segment may thus be defined. A straight line is then produced from the first portion to the second portion and forms a specific angle to the axis of rotation.

In the case of excessive contact angles, the bearing however is no longer able to suitably take up the radially acting forces, and the efficiency of the bearing reduces. This affects in particular contact angles of more than 80°, such that the contact angle in some exemplary embodiments is selected to be smaller than this angle.

In a further embodiment the cap segment is embedded in a surface of a cylinder or a cylinder sleeve. Here, the cap segment is preferably formed in one of the two circular bases, i.e. not in the side of the cylinder. The cylinder or cylinder sleeve form has the advantage, amongst other things, that it can be easily connected to an inlet guide wheel or outlet guide wheel or a rotor.

In a further embodiment the cap segment is made of the same material as the ball segment. In this case not only the ball segment, but also the cap segment or the cylinder sleeve housing the cap segment or the cylinder absorbs the heat created as a result of the friction and transfers it to the fluid flowing around the bearing on account of the good thermal conductivity of the diamond. The portion along which the heat preferably shall be delivered can be set accordingly by the length (i.e. the length along the cylinder axis perpendicular to the cut surface) for example of the cylinder or the cylinder sleeve.

In a further embodiment the blood pump comprises a further mechanical bearing with a further ball segment and a further cap segment. This further mechanical bearing is often arranged at an opposite end of the rotor as considered along the axis of rotation of the rotor. The further mechanical bearing may be constructed similarly to the bearing described here. However, it may also have a larger radius of curvature or a smaller or identical radius of curvature as compared to the first-mentioned mechanical bearing.

In a further variant, for the case that the further mechanical bearing is mounted downstream, it is provided that the radius of curvature of the ball segment or cap segment is greater than the bearing arranged upstream. This may have a positive effect on the one hand on the transporting away of heat by the fluid and on the other hand on the flow properties of the fluid.

In a further embodiment the ball segment is connected to a journal, for example to a metal or ceramic pin, wherein this use is provided for example by means of an adhesive material, such as an epoxy resin. The journal is preferably embedded in a metal element, for example a tubular metal element, which is formed as part of an inlet guide wheel or outlet guide wheel or a rotor. An example of an epoxy resin of this kind is the product EPO TEK® 301-2 from the company Epoxy Technology. This, however, is merely an example of a biocompatible and non-toxic adhesive. In essence, other biocompatible materials according to international standard ISO 10993 can also be used.

The journal simplifies the connection between the ball segment and the actual rotor. Similarly to a connecting pin, the journal brings about an improved and longer lasting connection between the rotor and the actual ball segment. The journal, amongst other things, ensures a concentricity of the bearing, wherein the journal is connected to the ball segment in such a way that the axis of rotation of the journal runs through a centre point of the ball segment. The centre point of the ball segment corresponds here to the point forming the centre point of a sphere and at which the sphere has the same radius as the ball segment. The journal connected to the ball segment is connected to the rotor in such a way that the axis of rotation of the journal runs coaxially with the rotor axis. Here, the journal is preferably connected to the ball segment for conjoint rotation so as not to generate any undesirable bearing friction between the ball segment and the journal. The prevention against rotation may be used additionally or alternatively to an integrally bonded connection of the ball segment to the journal and for example can be produced by means of a force-locked and/or form-locked connection.

In order to ensure an improved thermal conductivity, the journal can be embedded for example in a metal element or a ceramic element. The embedding metal element or ceramic element (such as tungsten carbide, diamond, copper) is in this case a cylinder sleeve or a tubular element which surrounds the journal. In one embodiment the embedding element can be made of titanium, for example.

Similarly to the ball segment, the cap segment, in particular if this is formed as a cylinder sleeve, can also be connected to a journal, so as to better anchor the cap segment in the pump. This journal may also be embedded by a metal or ceramic element.

In a further embodiment the ball segment is connected to a further cap segment, made of a diamond, for conjoint rotation. Here, the ball segment is connected to the cap segment in such a way that the surface of the ball segment not covered by the cap segment connected for conjoint rotation engages with the cap segment of the mechanical bearing and together therewith forms the frictional area. Due to the connection for conjoint rotation to a cap segment, which for example is formed similarly to the cap segment of the mechanical bearing, an even better dissipation of heat is ensured.

In a further embodiment the blood pump is formed in such a way that the mechanical bearing is designed such that the friction value between the ball segment and the cap segment is less than p=0.1, preferably p<0.05.

In a further embodiment the ball segment is covered by a plurality of microstructures. These microstructures on the one hand should not adversely affect the large-area contact between the cap segment and the ball segment, but should create small pockets which improve or simplify the elastic lubrication. The microstructures can be formed for example—similarly to a golf ball—as dimples or spiralled grooves, loxodromes or arcuate grooves.

In a further embodiment the ball segment is connected to the journal and/or the metal element by means of an engraved setting or bezel setting. Bezel settings or engraved settings are known in particular in the jewelry industry. The journal (or the metal element or a cylinder sleeve preferably made of hard material) comprises a recess, into which a portion of the ball segment is placed, and the ball segment is held in a frictionally engaged and/or form-locked manner by a collar (or bezel) of the journal (or of the metal element or the cylinder sleeve). In a further embodiment both the journal and the metal element or the cylinder sleeve have a recess in which the ball segment is bound. In this and the previous embodiment, the ball segment protrudes from the journal or the metal element or the cylinder sleeve in such a way that a surface of the ball segment together with the corresponding cap segment can form the bearing. The bezel setting or engraved setting for holding the ball segment in the metal element or in the journal can be provided with the additional use or without use of further joining means, such as adhesives, or a connection between the ball segment and the metal element, or the cylinder sleeve or the journal. In the embodiment free from joining means, the connection between the ball segment in the journal of the cylinder sleeve is thus without gaps, such that the heat transfer between the two elements is improved. Further setting forms which enable a gap-free connection between the ball segment in the journal are likewise suitable.

In a further embodiment in which the used hard material has an anisotropic hardness (i.e. a hardness which is different along different crystal lattice directions of the hard material), the ball segment is fastened to the axis of rotation in such a way that it is oriented with the greatest hardness parallel to the axis of rotation of the bearing. The opposite cap segment may also be oriented alternatively or additionally to the ball segment. An anisotropy occurs in particular when the used hard material is a monocrystal, such that a ball segment or a cap segment made of a monocrystalline hard material is preferred. In particular, a monocrystalline diamond has many of the desired properties. This can be arranged in the bearing in various embodiments in such a way that the “110” lattice plane of the diamond structure is used as a friction partner.

In a further embodiment the ball and/or cap segment is constructed from a polycrystalline hard material, such as SiC (silicon carbide), CBN (cubic boron nitride), PCD (polycrystalline diamond), PCR (polycrystalline ruby). The grain size is between 0.5 μm and 50 μm, preferably between 1 μm and 30 μm, particularly preferably between 2 μm and 20 μm (according to DIN ISO 6106:2015-11).

Further embodiments can be inferred from the following exemplary embodiments. In the drawings:

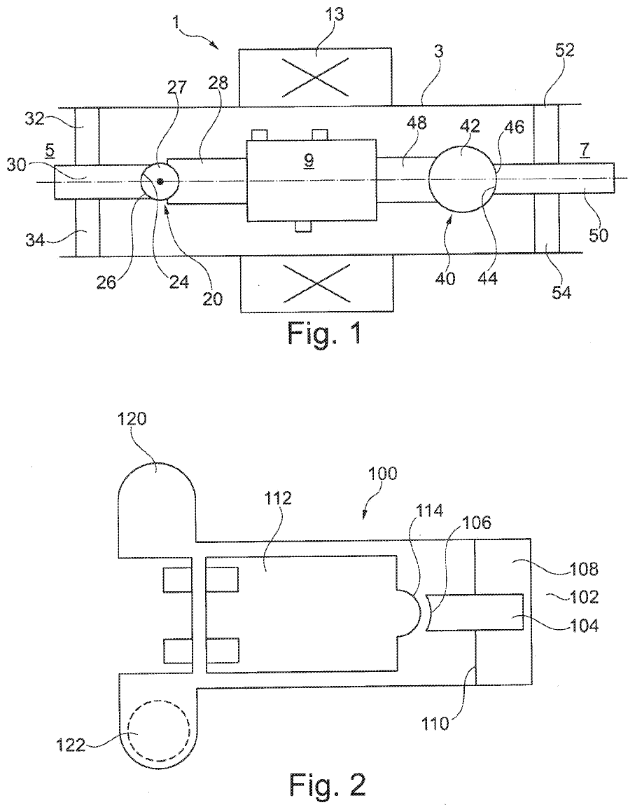

FIG. 1 shows a schematic depiction of an axial pump; FIG. 2 shows a schematic depiction of an axial pump with a tangential outlet;

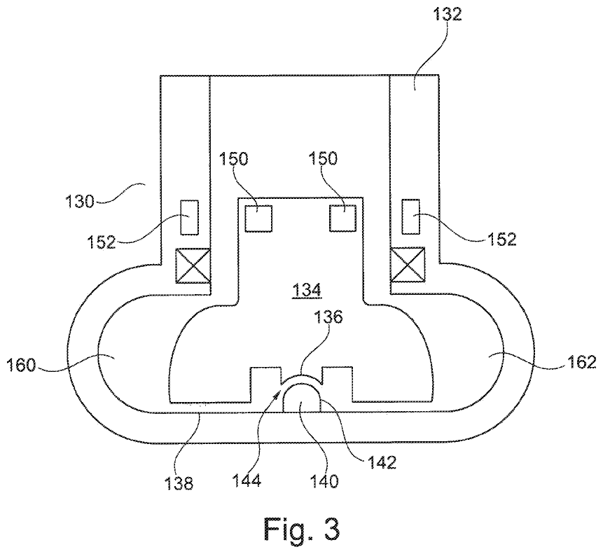

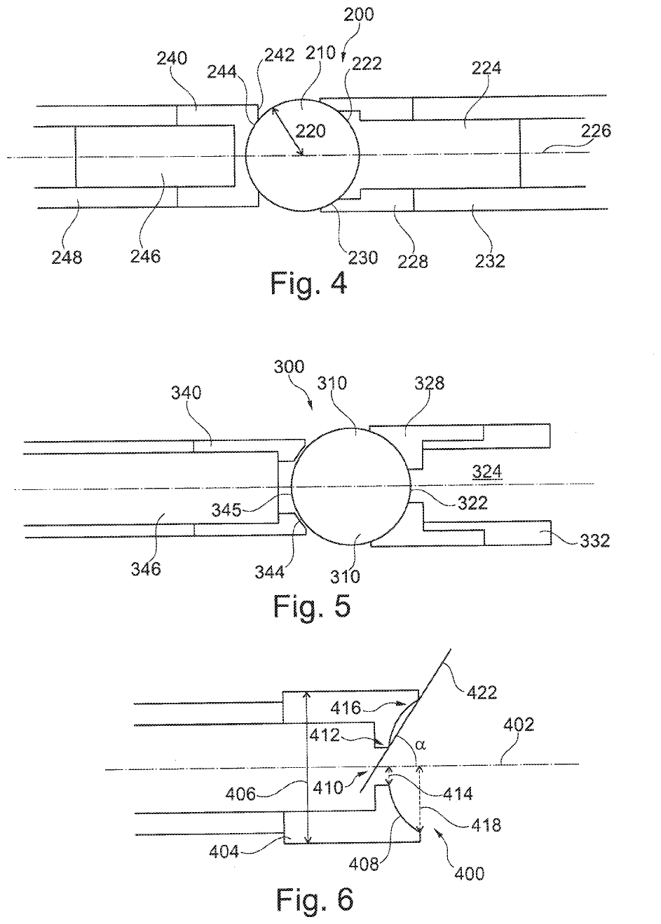

FIG. 3 shows a schematic depiction of a further blood pump; FIG. 4 shows a variant of a mechanical bearing;

FIG. 5 shows a further variant of a mechanical bearing;

FIG. 6 shows an illustration of a contact angle;

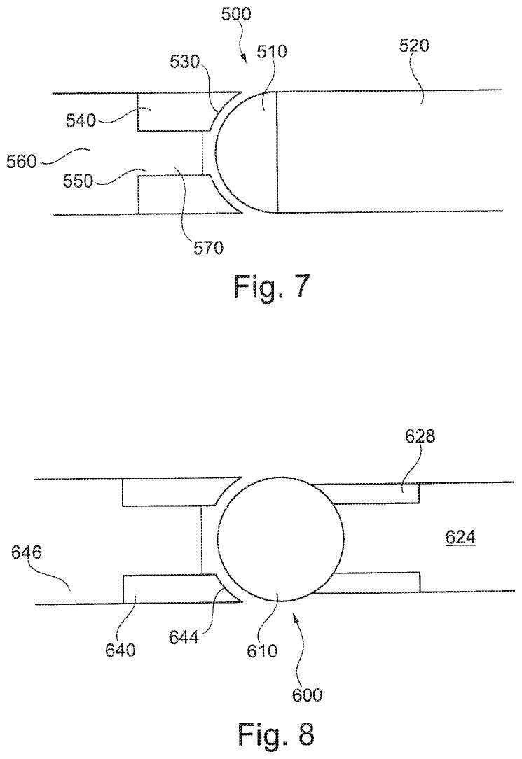

FIG. 7 shows a further embodiment of a bearing assembly;

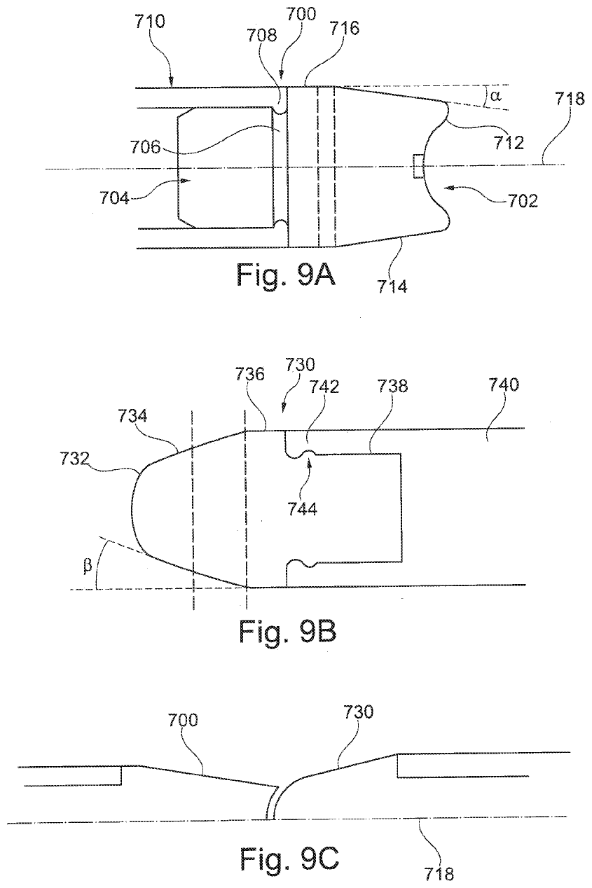

FIG. 8 shows a further embodiment of a bearing assembly; and FIG. 9 shows a further embodiment of a bearing assembly.

FIG. 1 shows an exemplary blood pump. Although this is an axial pump in the present example, with an inlet and outlet arranged along an axis of rotation, the blood pump may also be an axial pump with radial outlet or tangential outlet, wherein the rotor is arranged either also in the cylindrical inlet or also in the tangential outlet. Furthermore, the bearing design presented here may also be used in radial pumps.

The blood pump 1 comprises a housing 3 with an upstream inlet 5 and a downstream outlet 7. A rotor 9, which has a blading 11, in this case a spiral, is disposed between the inlet 5 and the outlet 7. The rotor houses, in its interior, permanent magnets which can be set in rotation by means of a stator 13 arranged externally on the housing 3. The rotor 11 also has an axis of rotation 15, which here is substantially coincident with the axis of the cylindrical housing 3.

The blood pump also comprises a first mechanical bearing 20, which has a ball segment surface 24 formed as a sphere 22, which corresponds with a cap segment-like surface 26. The sphere 22 is connected to the rotor for conjoint rotation via a rib. The cap segment 26 is formed as part of an inlet guide wheel 30 and constitutes a depression in the surface thereof. The inlet guide wheel 30 is in turn held in the housing via ribs 32 and 34. The pump also comprises a second mechanical bearing 40, which is of similar construction. In other words, the bearing comprises a ball segment 44 formed as part of a sphere 42 and corresponding to a cap segment 46. The sphere is in turn connected via a rib 48 to the rotor, whereas the cap segment 46 has been formed as part of an outlet guide wheel 50 on the surface thereof. The outlet guide wheel is also held in the lumen of the housing via ribs 52 and 54.

In the embodiment shown here, the front and the rear mechanical bearings, i.e. the first mechanical bearing and the second mechanical bearing, take up both axial forces, i.e. forces along the axis of rotation 15, and radial forces, i.e. forces perpendicular to the axis of rotation 15.

As mentioned at the outset, the bearing may also be used in other pumps. This is explained with reference to the alternative pump illustrated in FIG. 2. The blood pump 100 is an axial pump with a tangential outlet. The inlet 102 houses, amongst other things, an inlet guide wheel 104, which comprises a cap segment 106 arranged on an extension of the inlet guide wheel. The inlet guide wheel 104 is held within the housing via ribs 108 and 110. The rotor 112, besides a blading and permanent magnets which set the rotor in rotation by means of a stator, also comprises, at an upstream end of the rotor, a ball segment 114, which corresponds to the cap segment 106. In its downstream end, the rotor 112 is mounted passively by means of a permanent magnetic bearing. A spiral chamber 120 running around the rotor tangentially has an outlet 122, which is downstream of the inlet 102. In the present exemplary embodiment the pump is a pump formed with merely one mechanical bearing, which additionally has a downstream radial passive magnetic bearing.

FIG. 3 shows a further variant of a blood pump with a hard-material bearing according to the application. The pump 130 comprises a housing 132, in which a rotor 134 is mounted. The rotor sketched in FIG. 3 may comprise either a radial blading and/or an axial blading. The rotor 134 has, on its downstream rear side, an indentation or recess, in which the cap segment 136 of a mechanical bearing is arranged. On the wall 138 of the housing 132 opposite the rear side, there is an extension 140, which at its upstream end comprises a ball segment 142, which corresponds with the cap segment 136 of the rotor. In an alternative embodiment the ball segment is arranged on the rotor and the cap segment is arranged in the housing.

Besides the mechanical bearing 144 which comprises the cap segment 136 and the ball segment 142, permanent magnets 150 are arranged at the upstream end of the rotor 134. The permanent magnets 150, together with a ring magnet or a plurality of permanent magnets 152, form a passive magnetic bearing which can take up radial forces and/or axial forces. In the case of an axial bearing the poles of the permanent magnets are oriented parallel to the axis of rotation. In the case of a radial bearing the poles may also be oriented perpendicular to the axis of rotation.

In this embodiment there is no need in some variants for an upstream (i.e. entry-side) inlet guide wheel. Furthermore, it is also possible to dispense with a downstream outlet guide wheel. In the shown variant there is no inlet or outlet guide wheel provided. Regardless of the passive magnetic bearing, the rotor may comprise further permanent magnets, which are used for coupling to the motor stator 154.

The outlet 160 comprises a spiral chamber or volute 162, such that the blood in some exemplary embodiments can flow out tangentially to the inlet direction.

An embodiment of a mechanical bearing will be explained in greater detail with reference to FIG. 4. The bearing 200 comprises a sphere 210 which has a radius 220 of 1 mm. The sphere 210 is made of a diamond which has been produced by means of a CVD process. The sphere 210 is connected to a ceramic journal 224 by means of an epoxy adhesive 222. The ceramic journal here has a length of 1 cm and is for example anchored in an axis of the rotor. The length extends along the axis of rotation 226. A cylinder sleeve 228 with a spherical zone-shaped portion 230 is also provided, wherein the spherical zone portion 230 has the same radius of curvature as the sphere 210. The cylinder sleeve-shaped portion 228 is likewise made of a diamond. A metal casing, which already constitutes part of the rotor, is situated adjacent to the cylinder sleeve.

The sphere 210 has a radius of curvature corresponding to the radius 220. Furthermore, the portion of the bearing 200 that is fixedly connected to the sphere 210 is directly coupled to the rotor. This means that as the rotor rotates, the sphere 210 turns about the axis of rotation 226. Since both the sphere 210 and the cylinder sleeve 228 are made of a diamond, this embodiment of the mechanical bearing has good heat dissipation properties. Since the cylinder sleeve 228 is connected to the sphere 210 likewise fixedly, i.e. in an integrally bonded manner, by the epoxy adhesive 222, the cylinder sleeve is connected to the sphere for conjoint rotation. The static part (i.e. the non-rotating part) of the mechanical bearing 200 comprises a further cylindrical sleeve 240, which at the cut surface 242 comprises a cap segment 244. The cap segment is formed here as a spherical dome, the radius of curvature of which matches the radius of curvature of the sphere 210. A journal 246 is inserted into an opening in the cylinder 240 and is fixedly connected thereto. The journal can be made for example of a ceramic or of a metal, such as titanium. A metal casing 248, which for example may be part of an inlet guide wheel, is disposed adjacently to the cylinder 240. Furthermore, the metal sleeve 248 may also be part of an outlet guide wheel. The sphere 210 consists of a monocrystalline diamond and in an alternative embodiment as compared to that described here is held in the metal cylinder sleeve 228 by means of an engraved setting. The gap between the sphere 210 and the journal 224 can hereby be reduced or completely avoided, such that the transfer of heat between these elements is improved. The sphere is held in the cylinder sleeve in such a way that the “110” plane of the diamond structure is perpendicular to the axis of rotation 226.

A further embodiment of a mechanical bearing is shown in FIG. 5. The bearing of FIG. 5 likewise comprises a sphere 310 with a radius 320, which in the present case is 3 mm. The sphere is glued to a ceramic journal 324 in a region 322. The ceramic journal has a length of 0.5 cm. The sphere 320 is also framed by a cylindrical sleeve 228, which in the present exemplary embodiment is made of a ceramic. The sphere 310 is made of a diamond. The sleeve 332 is part of the rotor and is connected thereto for conjoint rotation. The static part of the mechanical bearing comprises a cylinder 340, which has a cap segment 344, and also has an opening 345 such that a gap 347 is situated between the journal 346 and the sphere 310. This gap may have hydrodynamic advantages. Since, however, the sphere 310 only has a very short distance from the cap segment 344, for example a distance of 5 μm, merely an elastohydrodynamic lubrication is provided in the region of the contact area between the sphere 310 and the cap segment 344. The sphere 310 and/or the cap segment 344 are/is made of a polycrystalline hard material with a mean grain size of 10 μm, wherein the actual grain sizes lie between 5 μm and 13 μm.

The contact area, which is defined substantially by the cap segment 344, is in the present case for example 2 mm². In the exemplary embodiment of FIG. 4 the contact area is much greater and for example may be 4 mm². The size, however, is determined substantially by the size of the sphere or the ball segment and the width of the spherical zone or the spherical dome of the cap segment.

A static part of a mechanical bearing 400 extending along an axis of rotation 402 is shown in FIG. 6. The static part comprises a cylinder 404, which has a diameter 406 of 2 mm. The cap segment 408 has a circular cut-out 410 which has a diameter of 0.5 mm. In the longitudinal section shown here (the other presentations of this application are also longitudinal sections along the axis of rotation 402), the cap segment starts at the point 412, which is arranged at a distance 414 perpendicular to the axis of rotation 402. The cap segment ends at the point 416, which is arranged at a distance 418 from the axis of rotation 402. A straight line 422 is defined by the points 412 and 416 and forms an angle α with the axis of rotation. The distance 414, as already mentioned, is 0.025 mm. The distance 418 is 0.9 mm. The radius R of the cap segment 408 corresponds to that of a sphere with a radius of 1.1 mm. The surface of the spherical zone can thus be calculated from the distances 414, 418 and the radius in accordance with the formula O=π(2 R H), wherein the height H can be calculated using Pythagoras' theorem on the basis of the fact that the distances are perpendicular to the axis of rotation. The radial projection is given from the height multiplied by twice the distance 414 times π. The axial projection is given from the circular area which is determined by the square of the greater distance 418 times π.

Further details of a mechanical bearing will be discussed with reference to FIGS. 7 and 8. For example, FIG. 7 shows a mechanical bearing 500 with a ball segment 510, which is designed in the form of a hemisphere. The flat surface of the hemisphere 510 is glued to a journal 520 for conjoint rotation. The static part of the bearing comprises a cap segment 530, which is formed as part of the cut surface of a cylinder sleeve 540. An opening 550 is formed in the cylinder sleeve, which opening is engaged by a journal 560 with an extension 570. The ball segment, which is formed as a hemisphere, has a radius corresponding to the radius of the cap segment 530. The hemisphere is made of a diamond. The cylinder sleeve 570 with the cap segment 530 can likewise be produced from a diamond, a ceramic, or a metal. Here, it is particularly advantageous if the ceramic or the metal for example is coated by a diamond-like carbon layer (DLC).

The embodiment of a mechanical bearing 600 shown in FIG. 8 corresponds substantially to that of the bearing shown in FIG. 4. The rotating part thus comprises a sphere 610 which is connected to a journal 624.

The sphere is also connected to a cylinder sleeve 628, which is made of diamond, for conjoint rotation. In a manner corresponding to the rotating part of the mechanical bearing, the static part of the bearing has a cylindrical sleeve 640 with a cap segment 644. The cylinder sleeve 640 is held by a journal 646, which in the present example is fabricated from titanium.

In FIGS. 9A to 9C a further variant of a bearing assembly is shown. FIG. 9A shows the bearing portion with a cap segment, FIG. 9B shows the portion with a ball segment, and FIG. 9C shows the two portions in engagement, as would be the case for example during blood pump operation.

The bearing pin 700 shown in FIG. 9A comprises, besides the cap portion 702, a journal 704. The journal and the cap portion are fabricated integrally and from SiC in the present example. Alternatively, the bearing pin may also be produced from another hard material mentioned in this application. The bearing pin 700 or the journal 700 has a groove 706, in which a collar 708 of a bearing sleeve 710 engages. This type of setting in the bearing sleeve, which may also be made of SiC, is an engraved setting between the cylindrical bearing sleeve and the bearing pin. In this way, the bearing pin 700 can be held in the bearing sleeve 710 in a manner in which rotation is prevented. The bearing sleeve may be an independent component, but may also be formed by a portion of the rotor or of a pump housing. The independent component may be connected to further components of the pump for example by means of a force-looked connection.

The cap portion 702, besides the actual cap 712, comprises a conical portion 714, the diameter of which widens towards the journal and ends in a short cylindrical portion 716. The conical portion runs at an angle α, which may be between 5° and 30°, preferably is between 9° and 15°, relative to the axis of rotation 718. The transition between the cylindrical portion 716 and the conical portion 714 is rounded here, such that there are no sharp edges. In other exemplary embodiments the two portions may also transition into one another with a kink, such that turbulences can be promoted at the edge.

The actual cap segment 712 has a constant radius of curvature or a radius of curvature widening from the centre of rotation radially outward. A recess 720 can be arranged in the middle of the cap segment 712 and can be used to provide additional lubrication of the bearing. The recess can be formed as a blind bore or as a through-hole through the entire bearing pin.

FIG. 9B shows the bearing pin 730 corresponding to the bearing pin 700 with a spherical cap 732, a conical portion 734, a cylindrical portion 736 and a journal 738. The bearing pin 730 is formed in one piece and is made of a polycrystalline diamond or—corresponding to the hard material of the bearing pin 700—of an SiC. The bearing pin 730 is held in a bearing sleeve 740. The bearing sleeve has a peripheral groove 742, in which a bead 744 of the journal 738 engages. This variant of the setting is a special form of the engraved setting, here an inverted engraved setting. The radius of curvature of the ball segment 746 of the spherical cap 732 is the same as or smaller than the radius of curvature of the cap segment 712 of the bearing pin 700. At the transition between the spherical cap 732 and the conical portion 734, there is a curved transition. At this transition the diameter of the bearing pin 730 is greater than the diameter of the bearing pin 700 in the region of the transition thereof between the cap segment 712 and the conical portion 714. The opening angle β of the cone in the present exemplary embodiment is greater than the opening angle α of the bearing pin 700 and is between 10° and 45°, preferably between 15° and 20°. The transition between the conical portion 734 and the cylindrical portion 736 is likewise rounded.

In FIG. 9C the bearing pin 700 and the bearing pin 730 are shown in engagement, and the proportions are shown qualitatively in a view radially from the axis of rotation 718. The length of the journal is shorter here in some embodiments than the remaining portions of each bearing pin together, but may also be longer.

Further exemplary embodiments will be obvious to a person skilled in the art.

To clarify the use of and to hereby provide notice to the public, the phrases “at least one of <A>, <B>, . . . and <N>” or “at least one of <A>, <B>, <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, or N including any one element alone or the one element in combination with one or more of the other elements which may also include, in combination, additional elements not listed. Unless otherwise indicated or the context suggests otherwise, as used herein, “a” or “an” means “at least one” or “one or more.”

1. A blood pump comprising:
a housing with an upstream inlet, a downstream outlet, and a rotatably mounted rotor with an axis and a blading; and
a mechanical bearing for supporting the rotor, the mechanical bearing comprising at least one ball segment and at least one cap segment which receives the at least one ball segment at least in part,
wherein the at least one ball segment and the at least one cap segment are rotationally symmetrical about an axis of rotation, at least in sections, and
wherein the at least one ball segment consists of a hard material selected from the group consisting of:
a diamond;
a nitride including a cubic crystalline boron nitride, a titanium nitride, a silicon nitride, or any other nitride;
a carbide including a silicon carbide, a boron carbide, a tungsten carbide, a vanadium carbide, a titanium carbide, a tantalum carbide, or any other carbide; and
an oxide including an aluminum oxide, a zirconium dioxide, or any other oxide.

2. The blood pump of claim 1, wherein the at least one ball segment is connected to a journal and the journal is embedded in a metal element.

3. The blood pump of claim 1, wherein the at least one ball segment is connected to or held by a journal, a metal element, or a cylinder sleeve made of hard material by means of a bezel setting and/or an engraved setting.

4. The blood pump of claim 1, wherein the at least one cap segment is connected to a journal and the journal is embedded in a metal element.

5. The blood pump of claim 1, wherein the at least one ball segment is connected to a further cap segment, made of a diamond, for conjoint rotation.

6. The blood pump of claim 1 further comprising a further mechanical bearing with a further ball segment and a further cap segment, wherein the further ball segment and/or the further cap segment are/is made of the same material as the at least one ball segment.

7. The blood pump of claim 1, wherein the further ball segment has a greater radius of curvature than the at least one ball segment.

8. The blood pump of claim 1, wherein the further ball segment has the same radius of curvature as the at least one ball segment.

9. (canceled)

10. The blood pump of claim 1, wherein the hard material is a monocrystalline diamond having a diamond structure, and the monocrystalline diamond is arranged in such a way that a 110 plane of the diamond structure is perpendicular to the axis of rotation.

11. The blood pump of claim 1, wherein the hard material is a polycrystalline hard material with a grain size of 2 to 20 μm.

12. The blood pump of claim 1, wherein the at least one ball segment is a sphere.

13. The blood pump of claim 1, wherein the at least one ball segment is a spherical segment.

14. The blood pump of claim 1, wherein the at least one cap segment, between a first distance and a second distance from the axis of rotation has a radius of curvature corresponding to a radius of curvature of the ball segment.

15. The blood pump of claim 14, wherein, in a longitudinal section of the at least one cap segment as considered along the axis of rotation, the first distance defines a first point of contact and the second distance defines a second point of contact between the at least one cap segment and ball segment, and a straight line defined by the first and second points of contact has an angle of more than 50° relative to the axis of rotation.

16. The blood pump of claim 1, wherein the at least one cap segment is made of the same material as the ball segment.

17. The blood pump of claim 1, wherein the at least one cap segment is made of a different material as compared to the ball segment.

18. The blood pump of claim 1, wherein the mechanical bearing is designed in such a way that a friction value between the at least one ball segment and the at least one cap segment is less than 0.2.

19. The blood pump of claim 1, wherein the at least one cap segment is formed as part of a surface of a cylinder or of a cylinder segment.

20. The blood pump of claim 1, wherein the at least one ball segment has a plurality of microstructures.
